(19) United States
(12) Patent Application Publication
(10) Pub. No.: US 2011/0021101 A1
(43) Pub. Date: Jan. 27, 2011

(54) MODIFIED STARCH BASED BINDER

(76) Inventors: Christopher M. Hawkins, Alexandria, OH (US); Liang Chen, New Albany, OH (US); Jesus M. Hernandez-Torres, Pataskala, OH (US); William E. Downey, Granville, OH (US)

Correspondence Address: MacMillan, Sobanski & Todd, LLC, One Maritime Plaza, Fifth Floor, 720 Water Street, Toledo, OH, 43604, US

(21) Appl. No.: 12/825,375

(22) Filed: Jun. 29, 2010

Related U.S. Application Data
(60) Provisional application No. 61/221,298, filed on Jun. 29, 2009.

(51) Int. Cl. C08F 251/00; C07K 1/107; D04H 13/00

(52) U.S. Cl. 442/327; 527/300; 527/312; 527/311; 527/200

(57) ABSTRACT

An aqueous binder composition is provided that includes a modified starch and a silane coupling agent, and optionally, a crosslinking agent. The starch from which the modified starch is derived is natural in origin, biorenewable, and is derived from plant sources. The modified starch has been chemically modified from its natural form and may have a degree of polymerization from about 20 to about 4000. Additionally, the modified starches have a low viscosity and cure at moderate temperatures. Advantageously, the modified starches are water dispersible and have excellent resistance to water after curing. In addition, the binder has a light color after being cured. The crosslinking agent may be any compound suitable for crosslinking the starch based compound. In exemplary embodiments, the silane coupling agent is an aminosilane. The environmentally friendly, biorenewable binder may be used in the formation of insulation materials and non-woven chopped strand mats.

### TECHNICAL FIELD AND INDUSTRIAL APPLICABILITY OF THE INVENTION

The present invention relates generally to rotary fiber insulation and non-woven mats, and more particularly, to a binder for use in manufacturing both fiberglass insulation and non-woven mats that is starch based, contains no added formaldehyde, and is environmentally friendly.

### BACKGROUND OF THE INVENTION

Conventional fibers are useful in a variety of applications including reinforcements, textiles, and acoustical and thermal insulation materials. Although mineral fibers (e.g., glass fibers) are typically used in insulation products and non-woven mats, depending on the particular application, organic fibers such as polypropylene, polyester, and multi-component fibers may be used alone or in combination with mineral fibers in forming the insulation product or non-woven mat.

Fibrous insulation is typically manufactured by fiberizing a molten composition of polymer, glass, or other mineral and spinning fine fibers from a fiberizing apparatus, such as a rotating spinner. To form an insulation product, fibers produced by the rotating spinner are drawn downwardly from the spinner towards a conveyor by a blower. As the fibers move downward, a binder material is sprayed onto the fibers and the fibers are collected into a high loft, continuous blanket on the conveyor. The binder material gives the insulation product resiliency for recovery after packaging and provides stiffness and handleability so that the insulation product can be handled and applied as needed in the insulation cavities of buildings. The binder composition also provides protection to the fibers from interfilament abrasion and promotes compatibility between the individual fibers.

The blanket containing the binder-coated fibers is then passed through a curing oven and the binder is cured to set the blanket to a desired thickness. After the binder has cured, the fiber insulation may be cut into lengths to form individual insulation products, and the insulation products may be packaged for shipping to customer locations. One typical insulation product produced is an insulation batt or blanket, which is suitable for use as wall insulation in residential dwellings or as insulation in the attic and floor insulation cavities in buildings. Another common insulation product is air-blown or loose-fill insulation, which is suitable for use as sidewall and attic insulation in residential and commercial buildings as well as in any hard-to-reach locations. Loose-fill insulation is formed of small cubes that are cut from insulation blankets, compressed, and packaged in bags.

Non-woven mats may be formed by conventional wet-laid processes. For example, wet chopped fibers are dispersed in a water slurry that contains surfactants, viscosity modifiers, defoaming agents, and/or other chemical agents. The slurry containing the chopped fibers is then agitated so that the fibers become dispersed throughout the slurry. The slurry containing the fibers is deposited onto a moving screen where a substantial portion of the water is removed to form a web. A binder is then applied, and the resulting mat is dried to remove any remaining water and cure the binder. The formed non-woven mat is an assembly of dispersed, individual glass filaments.

Various attempts have been made to reduce undesirable formaldehyde emissions from formaldehyde-based resins. For example, various formaldehyde scavengers such as ammonia and urea have been added to the formaldehyde-based resin in an attempt to reduce formaldehyde emission from the insulation product. Because of its low cost, urea is added directly to the uncured resin system to act as a formaldehyde scavenger. The addition of urea to the resin system produces urea-extended phenol-formaldehyde resole resins. These resole resins can be further treated or applied as a coating or binder and then cured. Unfortunately, the urea-extended resoles are unstable, and because of this instability, the urea-extended resoles must be prepared on site. In addition, the binder inventory must be carefully monitored to avoid processing problems caused by undesired crystalline precipitates of dimer species that may form during storage. Ammonia is not a particularly desirable alternative to urea as a formaldehyde scavenger because ammonia generates an unpleasant odor and may cause throat and nose irritation to workers. Further, the use of a formaldehyde scavenger in general is undesirable due to its potential adverse affects to the properties of the insulation product, such as lower recovery and lower stiffness.

In addition, previous arts have focused on the use of polyacrylic acid with a polyhydroxy crosslinking agent or carbohydrate-based chemistry that is linked to the Maillard reaction. Polyacrylic acid inherently has problems due to its acidity and associated corrosion of machine parts. In addition, polyacrylic acid binders have a high viscosity, high curing temperatures, and high associated curing costs. Further, the Maillard-based products have an undesirable dark brown color after curing. Also, the use of large amounts of ammonia needed to make the binder presents a safety risk and possible emission problems.

Alternative polymeric binder systems to those described above for fibrous glass products have also been proposed. However, these alternative binder systems remain problematic. For example, low molecular weight, low viscosity binders which allow maximum vertical expansion of the insulation pack in the transfer zone generally cure to form a non-rigid plastic matrix in the finished product, thereby reducing the attainable vertical height recovery of the finished insulation product when installed. Conversely, high viscosity binders, which generally cure to form a rigid matrix in the finished product, do not allow the desired maximum vertical expansion of the coated, uncured pack.

In view of the existing problems with current binders, there remains a need in the art for a binder system that does not corrode machine parts, does not emit formaldehyde, and which is environmentally.

### SUMMARY OF THE INVENTION

It is an object of the present invention to provide a binder composition for use in the formation of fiberglass insulation and non-woven chopped strand mats that includes at least one modified starch that is natural in origin and has a degree of polymerization from about 20 to about 4000 and at least one silane coupling agent. The modified starch may be derived from a plant source selected from corn, potatoes, soybeans, rice, beets, sugar cane, cassava, and mixtures thereof. In addition, the modified starch may have a viscosity less than about 205 cps at 9% solids. Optionally, the binder composition includes a crosslinking agent, a dust suppression agent, a cure accelerator, a pH adjusting agent, and/or a moisture resistant agent. The pH adjusting agent may adjust the pH of the binder composition to a pH from about 1 to about 6. The binder composition is free of added formaldehyde and is environmentally friendly.

It is another object of the present invention to provide a fibrous insulation product that includes a plurality of randomly oriented fibers and a binder composition applied to at least a portion of the fibers and interconnecting the fibers. The binder includes at least one modified starch that is natural in origin and has a degree of polymerization from about 20 to about 4000 and at least one silane coupling agent. The modified starch may have a viscosity less than about 205 cps at 9% solids. In exemplary embodiments, the modified starch is derived from a plant source selected from corn, potatoes, soybeans, rice, beets, sugar cane, cassava, and mixtures thereof. The binder composition may optionally include at least one member selected from a crosslinking agent, a dust suppression agent, a cure accelerator, a pH adjusting agent, and a moisture resistant agent. Further, a pH adjusting agent may be included in the binder composition to adjust the pH to a pH range from about 1 to about 6.

It is yet another object of the present invention to provide a non-woven chopped strand mat formed of a plurality of randomly oriented glass fibers having a discrete length enmeshed in the form of a mat having a first major surface and a second major surface and a binder composition at least partially coating the first major surface of the mat. The binder includes at least one modified starch that has a degree of polymerization from about 20 to about 4000 which is derived from natural sources and at least one silane coupling agent. Additionally, the binder may include at least one member selected from a crosslinking agent, a dust suppression agent, a cure accelerator, a pH adjusting agent, and a moisture resistant agent. In exemplary embodiments, the modified starch is derived from a plant source selected from corn, potatoes, soybeans, rice, beets, sugar cane, cassava, and mixtures thereof. The pH adjusting agent may be utilized to adjust the pH of the binder composition to a pH from about 1 to about 6. Unlike conventional formaldehyde compositions, the binder has a light color upon curing. In addition, the binder is environmentally friendly and free of added formaldehyde.

It is an advantage of the present invention that the modified starch is natural in origin and derived from renewable resources.

It is also an advantage of the present invention that the modified starch based binders are water dispersible and have excellent resistance to water after curing.

It is a further advantage of the present invention that the binder can be cured at temperatures lower than conventional formaldehyde-based binders, thereby reducing manufacturing costs and gaseous emissions.

It is yet another advantage of the present invention that the modified starch is readily available and is low in cost.

It is also an advantage of the present invention that insulation products and non-woven mats utilizing the inventive binder composition can be manufactured using current manufacturing lines, thereby saving time and money.

It is another advantage of the present invention that the binder composition has no added formaldehyde.

It is a feature of the present invention that the modified starch may have a degree of polymerization from about 20 to about 4000.

It is also a feature of the present invention that the modified starch may have a viscosity less than about 205 cps at 9% solids.

It is a feature of the present invention that the modified starch can form an aqueous mixture that can be applied by conventional binder applicators, including spray applicators.

It is a further feature of the present invention that the binder can be acidic, neutral, or basic.

It is also a feature of the present invention that the binder has a light color upon curing.

It is another feature of the present invention that the inventive insulation products and non-woven mats have no added formaldehyde.

It is also a feature of the invention that the inventive binder composition can be useful for composite reinforcements, such as chopped strands, for use in thermoplastics, thermosets, and roofing applications. In addition, the inventive binders may be used in both single and multi-end rovings.

The foregoing and other objects, features, and advantages of the invention will appear more fully hereinafter from a consideration of the detailed description that follows. It is to be expressly understood, however, that the drawings are for illustrative purposes and are not to be construed as defining the limits of the invention.

### BRIEF DESCRIPTION OF THE DRAWINGS

The advantages of this invention will be apparent upon consideration of the following detailed disclosure of the invention, especially when taken in conjunction with the accompanying drawings wherein:

FIG. 1 is a schematic illustration of the formation of a faced insulation product with a post-cure application of urea onto the insulation blanket prior to the application of a facing layer;

FIG. 2 is a is an elevational view of a manufacturing line for producing a fiberglass insulation product that does not contain a facing material according to at least one exemplary embodiment of the present invention; and

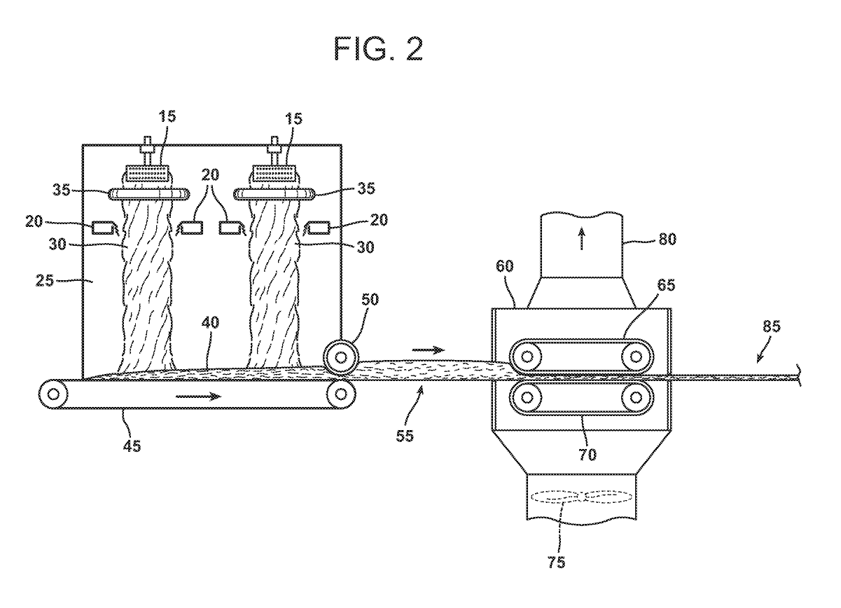

FIG. 3 is a schematic illustration of a wet-laid processing line for forming a chopped strand mat utilizing the inventive binder composition according to at least one exemplary embodiment of the present invention.

### DETAILED DESCRIPTION AND PREFERRED EMBODIMENTS OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein. All references cited herein, including published or corresponding U.S. or foreign patent applications, issued U.S. or foreign patents, and any other references, are each incorporated by reference in their entireties, including all data, tables, figures, and text presented in the cited references.

In the drawings, the thickness of the lines, layers, and regions may be exaggerated for clarity. It will be understood that when an element such as a layer, region, substrate, or panel is referred to as being “on” another element, it can be directly on the other element or intervening elements may also be present. Also, when an element is referred to as being “adjacent” to another element, the element may be directly adjacent to the other element or intervening elements may be present. The terms “top”, “bottom”, “side”, and the like are used herein for the purpose of explanation only. Like numbers found throughout the figures denote like elements. It is to be noted that the phrase “binder composition”, “binder mixture”, and “binder” may be used interchangeably herein.

The present invention relates to an aqueous binder composition that is starch based and environmentally friendly. In addition, the binder is free of added formaldehyde. The binder includes a modified starch and a silane coupling agent, and optionally, a crosslinking agent, a pH adjuster, a cure accelerator, a dust suppression agent, and/or a moisture resistant agent. Additionally, the binder has a light (e.g., white or tan) color after it has been cured. The binder may be used in the formation of insulation materials and non-woven chopped strand mats. The inventive binders may also be useful in forming particleboard, plywood, and/or hardboards.

In exemplary embodiments, the binder includes at least one modified starch that is obtained from natural sources and derived from renewable resources. For instance, the starch may be derived from plant sources such as corn, potatoes, soybean, rice, beets, sugar cane, and/or cassava, or from other plants that have a high starch content. The starch has been chemically modified from its naturally derived source, such as, for example, by oxidation, bleaching, or acid or base treatment. It is also considered to be within the purview of the invention to utilize a man-made (e.g., synthetic) starch in place of or in addition to the natural starches described herein. The modified starch may have a degree of polymerization from about 20 to about 4000, from about 100 to about 1000, or from about 200 to about 500. The chemical oxidation or modification of the starch permits the starch to react under high heat. In addition, the modified starches may have a viscosity from about 2 to about 330 cps, from about 5 to about 300 cps, from about 10 to about 205 cps, or from about 2 to about 75 cps at 9% solids. In some exemplary embodiments, the viscosity of the modified starch may be from about 45 to about 328 cps at 9% solids. Non-limiting examples of suitable starches for use in the instant invention in include Super Film® 227, a modified starch with a viscosity from 225-575 cps at 9% solids; Super Film® 233, a modified starch with a viscosity from 340-750 cps at 9% solids; Super Film® 235 and 235D, modified starches with a viscosity from 175-575 cps at 10% solids; Super Film® 244D, a modified starch with a viscosity from 150-475 cps at 13% solids; and Super Film® 270W, a modified starch with a viscosity from 200-625 cps at 22% solids. Each of the Super Film® modified starches identified above is commercially available from Cargill. The modified starch(es) may be present in the binder composition in an amount from about 50% to about 99% by weight of the total solids in the binder composition, from about 70% to about 95% by weight, from about 70% to about 90% by weight, or from about 80% to about 90% by weight. As used herein, % by weight indicates % by weight of the total solids in the binder composition.

Conventionally, starches do not have enough water resistance to be used effectively in a binder. Additionally, un-modified starches have a viscosity that is too high for use in a binder composition. It has been surprisingly discovered, however, that modified starches, such as those described above, are water dispersible and have excellent resistance to water after curing. Further, these modified starches beneficially have a low viscosity and cure at moderate temperatures (e.g., 80-200° C.) by itself or with additives. The low viscosity enables the modified starch to be utilized in a binder composition. In exemplary embodiments, the viscosity of the modified starches is less than about 205 cps at 9% solids. The use of modified starch in the inventive binder composition is advantageous in that modified starch is readily easily obtainable and is low in cost.

Another advantageous feature of the modified starch is the ability of the modified starch to bind or crosslink with itself. Prior to oxidation, starch contains primary and secondary alcohols. After oxidation, the modified starch contains approximately 10% of aldehydes and carboxylic acids that are able to react with the alcohols on the starch, thereby promoting self-crosslinking. More specifically, the oxidized starch contains nucleophilic alcohols and electrophilic carboxylic acids and aldehydes. The primary and secondary alcohols react with the electrophilic moieties of a crosslinking agent (e.g., carbonyls) while the carboxylic acids and aldehydes react with the nucleophilic moieties of a crosslinking agent (e.g., alcohols, amines, carbenes, etc.). The addition of a separate crosslinking agent such as citric acid, for example, assists in the crosslinking and the formation of covalent bonds.

Another component of the binder composition is a silane coupling agent. The silane coupling agent(s) may be present in the binder composition in an amount from about 0.01% to about 5.0% by weight of the total solids in the binder composition, from about 0.01% to about 2.5% by weight, or from about 0.01% to about 1.0% by weight. Examples of silane coupling agents that may be used in the binder composition may be characterized by the functional groups alkyl, aryl, amino, epoxy, vinyl, methacryloxy, ureido, isocyanato, and mercapto. In exemplary embodiments, the silane coupling agent(s) include silanes containing one or more nitrogen atoms that have one or more functional groups such as amine (primary, secondary, tertiary, and quaternary), amino, imino, amido, imido, ureido, or isocyanato. Specific, non-limiting examples of suitable silane coupling agents include, but are not limited to, aminosilanes (e.g., 3-aminopropyl-triethoxysilane and 3-aminopropyl-trihydroxysilane), epoxy trialkoxysilanes (e.g., 3-glycidoxypropyltrimethoxysilane and 3-glycidoxypropyltriethoxysilane), methyacryl trialkoxysilanes (e.g., 3-methacryloxypropyltrimethoxysilane and 3-methacryloxypropyltriethoxysilane), hydrocarbon trialkoxysilanes, amino trihydroxysilanes, epoxy trihydroxysilanes, methacryl trihydroxy silanes, and/or hydrocarbon trihydroxysilanes. In one or more exemplary embodiment, the silane is an aminosilane, such as γ-aminopropyltriethoxysilane.

In addition, the binder composition may contain at least one crosslinking agent. The crosslinking agent may be any compound suitable for crosslinking the modified starch. Non-limiting examples of suitable crosslinking agents include citric acid (and salts thereof, such as ammonium citrate or sodium citrate), polyacrylic acid (and salts thereof), polyacrylic acid resins such as QXRP 1734 and Acumer 9932 (a 46% solids polyacrylic acid), both commercially available from The Dow Chemical Company, triethanol amine, sodium metaborate, polyoxyalkyleneamines (e.g., Jeffamine®, amines commercially available from Huntsman Corporation), polyamines, glycerol, triethanolamine, polyols, polyacrylic acid, polycarboxylic acid, polycarboxylic acid with anhydride (i.e., mixed anhydrides), ammonium citrate, adipic acid, acetic anhydride, organic acids, inorganic acids, organic bases, inorganic bases, proteins, and combinations thereof. The crosslinking agent may be present in the binder composition in an amount from about 1.0% to about 30% by weight of the total solids in the binder composition, from about 5.0% to about 25% by weight, or from about 10.0% to about 20.0% by weight. In exemplary embodiments, the crosslinking agent is polyacrylic acid or citric acid (i.e., an electrophilic crosslinking agent) or triethanolamine, or glycerol (i.e., a nucleophilic crosslinking agent).

Additionally, the binder composition may include a cure accelerator and/or a catalyst. Cure accelerators and/or catalysts that may be used in the binder formulation include, but are not limited to, sodium hypophosphite, sodium phosphate, potassium phosphate, disodium pyrophosphate, tetrasodium pyrophosphate, sodium tripolyphosphate, sodium hexamethaphosphate, potassium phosphate, potassium tripolyphosphate, sodium trimetaphosphate, sodium tetramethaphosphate, or mixtures thereof. The cure accelerator and/or catalyst may be present in the binder composition in an amount from about 0% to about 15% by weight of the total solids in the binder composition, from about 0.5% to about 15.0% by weight, or from about 2.0% to about 10.0% by weight.

Further, the binder composition may contain a pH adjuster in an amount sufficient to adjust the pH to a desired level. The pH may be adjusted depending on the intended application, or to facilitate the compatibility of the ingredients of the binder composition. In exemplary embodiments, the pH adjuster is utilized to adjust the pH of the binder composition to an acidic pH. Examples of suitable acidic pH adjusters include mono- or polycarboxylic acids, such as, but not limited to, citric acid, acetic acid, and sulfuric acid, anhydrides thereof, and inorganic salts that can be acid precursors. The acid adjusts the pH, and in some instances, acts as a crosslinking agent, as discussed above. The pH of the binder composition, when in an acidic state, may range from about 1 to about 6, and in some exemplary embodiments, from about 1 to about 5. In at least one exemplary embodiment, the pH of the binder composition is about 3. It is to be appreciated that the final pH of the cured product has a pH that is greater (e.g., higher) than the pH of the binder composition.

In another embodiment of the invention, the pH adjuster has a basic pH and is added to the binder composition in an amount sufficient to produce a binder that has a desired, basic pH. Non-limiting examples of suitable basic pH adjusters include sodium bisulfate, sodium hydroxide, potassium hydroxide, and/or ammonium hydroxide. The pH of the binder composition, when in a basic state, may range from about 8 to about 14, or from about 8 to about 12. In at least one exemplary embodiment, the pH of the binder composition is about 9. It is to be appreciated that the binder may alternatively have a neutral pH.

The binder composition may also contain a moisture resistant agent, such as a alum, aluminum sulfate, latex, a silicon emulsion, a hydrophobic polymer emulsion (e.g., polyethylene emulsion or polyester emulsion), and mixtures thereof. In at least one exemplary embodiment, the latex system is an aqueous latex emulsion. The latex emulsion includes latex particles that are typically produced by emulsion polymerization. In addition to the latex particles, the latex emulsion may include water, a stabilizer such as ammonia, and a surfactant. The moisture resistant agent may be present in the binder composition in an amount from about 0% to about 20% by weight of the total solids in the binder composition, or from about 0.5% to about 5.0% by weight.

The binder may also include a dust suppression agent such as a mineral oil, vegetable oil, peanut oil, silicone, and the like. In exemplary embodiments, the dust suppression agent is present in the binder composition in an amount up to 40% by weight of the total solids in the binder composition.

The binder may optionally contain conventional additives such as, but not limited to corrosion inhibitors, dyes, pigments, fillers, colorants, UV stabilizers, thermal stabilizers, anti-foaming agents, anti-oxidants, emulsifiers, preservatives (e.g., sodium benzoate) and mixtures thereof. Other additives may be added to the binder composition for the improvement of process and product performance. Such additives include lubricants, wetting agents, surfactants, antistatic agents, and/or water repellent agents. Additives may be present in the binder composition from trace amounts (such as <about 0.1% by weight the binder composition) up to about 10.0% by weight of the total solids in the binder composition. In some exemplary embodiments, the additives are present in an amount from about 0.1% to about 5.0% by weight of the binder composition by weight of the total solids in the binder composition.

The binder further includes water to dissolve or disperse the active solids for application onto the reinforcement fibers. Water may be added in an amount sufficient to dilute the aqueous binder composition to a viscosity that is suitable for its application to the reinforcement fibers and to achieve a desired solids content on the fibers. In particular, the binder composition may contain water in an amount from about 70% to about 98.0% by weight of the total solids in the binder composition.

The binder composition may be made by dispersing the crosslinking agent in water to form a mixture. Next, the modified starch is mixed with the crosslinking agent in the mixture to form a stock mixture. If desired, a cure accelerator may be added to the stock mixture. The silane coupling agent is added to the stock mixture to form the binder composition. The binder composition may be further diluted with water to obtain a desired amount of solids. If necessary, the pH of the mixture may be adjusted to the desired pH level.

In the broadest aspect of the invention, the binder composition is formed of a modified starch (e.g., modified corn starch) and a silane coupling agent (e.g., aminosilane). The range of components used in the inventive binder composition according to embodiments of the invention is set forth in Table 1.

TABLE 1

| | % By Weight |
|---|---|
| Component | of Total Solids |
| Modified Starch | 95-99.9 |
| Coupling Agent | 0.1-5.0 |

Aqueous binder compositions according to other exemplary embodiments of the present invention that include a crosslinking agent (e.g., citric acid, glycerol, tri-sodium trimetaphosphate, etc.) are set forth in Table 2.

TABLE 2

| | % By Weight |
|---|---|
| Component | of Total Solids |
| Modified Starch | 50-99.8 |
| Coupling Agent | 0.1-5.0 |
| Crosslinking Agent | 0.1-50 |

Aqueous binder compositions according to further exemplary embodiments of the present invention are set forth in Table 3.

TABLE 3

| | % By Weight |
|---|---|
| Component | of Total Solids |
| Modified Starch | 50-99.8 |
| Coupling Agent | 0.1-5.0 |
| Crosslinking Agent | 0.1-50 |
| Cure Accelerator | 0-15 |
| Catalyst | 0-15 |
| Dust Suppressing Agent | 0.1-40 |
| pH Adjusting Agent | Amount |
| | Sufficient To |
| | Achieve |
| | Desired pH |

In one exemplary embodiment, the binder composition is used to form an insulation product. Fibrous insulation products are generally formed of matted inorganic fibers bonded together by a cured thermoset polymeric material. Examples of suitable inorganic fibers include glass fibers, wool glass fibers, and ceramic fibers. Optionally, other reinforcing fibers such as natural fibers and/or synthetic fibers such as polyester, polyethylene, polyethylene terephthalate, polypropylene, polyamide, aramid, and/or polyaramid fibers may be present in the insulation product in addition to the glass fibers. The term “natural fiber” as used in conjunction with the present invention refers to plant fibers extracted from any part of a plant, including, but not limited to, the stem, seeds, leaves, roots, or phloem. Examples of natural fibers suitable for use as the reinforcing fiber material include basalt, cotton, jute, bamboo, ramie, bagasse, hemp, coir, linen, kenaf, sisal, flax, henequen, and combinations thereof. Insulation products may be formed entirely of one type of fiber, or they may be formed of a combination of types of fibers. For example, the insulation product may be formed of combinations of various types of glass fibers or various combinations of different inorganic fibers and/or natural fibers depending on the desired application for the insulation. The embodiments described herein are with reference to insulation products formed entirely of glass fibers.

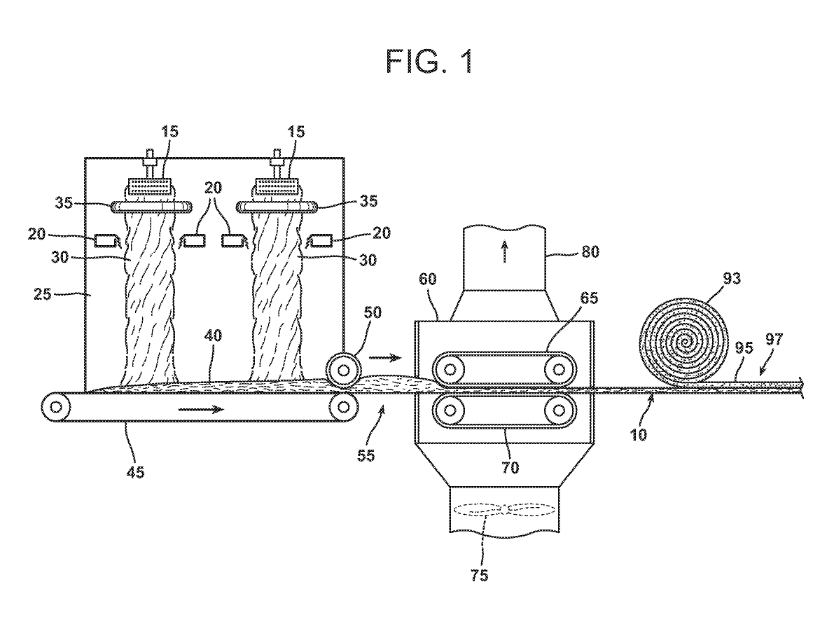

The manufacture of glass fiber insulation may be carried out in a continuous process by fiberizing molten glass, immediately forming a fibrous glass batt on a moving conveyor, and curing the binder on the fibrous glass insulation batt to form an insulation blanket as depicted in FIG. 1. Glass may be melted in a tank (not shown) and supplied to a fiber forming device such as a fiberizing spinner 15. The spinners 15 are rotated at high speeds. Centrifugal force causes the molten glass to pass through holes in the circumferential sidewalls of the fiberizing spinners 15 to form glass fibers. Glass fibers 30 of random lengths may be attenuated from the fiberizing spinners 15 and blown generally downwardly, that is, generally perpendicular to the plane of the spinners 15, by blowers 20 positioned within a forming chamber 25. It is to be appreciated that the glass fibers 30 may be the same type of glass or they may be formed of different types of glass. It is also within the purview of the present invention that at least one of the fibers 30 formed from the fiberizing spinners 15 is a dual glass fiber where each individual fiber is formed of two different glass compositions.

The blowers 20 turn the fibers 30 downward to form a fibrous batt 40. The glass fibers 30 may have a diameter from about 2 to about 9 microns, or from about 3 to about 6 microns. The small diameter of the glass fibers 30 helps to give the final insulation product a soft feel and flexibility.

The glass fibers, while in transit in the forming chamber 25 and while still hot from the drawing operation, are sprayed with the inventive aqueous binder composition by an annular spray ring 35 so as to result in a distribution of the binder composition throughout the formed insulation pack 40 of fibrous glass. Water may also be applied to the glass fibers 30 in the forming chamber 25, such as by spraying, prior to the application of the aqueous binder composition to at least partially cool the glass fibers 30. The binder may be present in an amount less than or equal to 25.0%, less than or equal to 20.0%, less than or equal to 15.0%, or less than or equal to 10.0% by weight of the total product. The low amount of binder contributes to the flexibility of the final insulation product.

The glass fibers 30 having the uncured resinous binder adhered thereto may be gathered and formed into an uncured insulation pack 40 on an endless forming conveyor 45 within the forming chamber 25 with the aid of a vacuum (not shown) drawn through the fibrous pack 40 from below the forming conveyor 45. The residual heat from the glass fibers 30 and the flow of air through the fibrous pack 40 during the forming operation are generally sufficient to volatilize a portion of the water from the binder before the glass fibers 30 exit the forming chamber 25, thereby leaving the remaining components of the binder on the fibers 30 as a viscous or semi-viscous high-solids liquid.

The coated fibrous pack 40, which is in a compressed state due to the flow of air through the pack 40 in the forming chamber 25, is then transferred out of the forming chamber 25 under exit roller 50 to a transfer zone 55 where the pack 40 vertically expands due to the resiliency of the glass fibers. The expanded insulation pack 40 is then heated, such as by conveying the pack 40 through a curing oven 60 where heated air is blown through the insulation pack 40 to evaporate any remaining water in the binder, cure the binder, and rigidly bond the fibers together. Heated air is forced though a fan 75 through the lower oven conveyor 70, the insulation pack 40, the upper oven conveyor 65, and out of the curing oven 60 through an exhaust apparatus 80. The cured binder imparts strength and resiliency to the insulation blanket 10. It is to be appreciated that the drying and curing of the binder may be carried out in either one or two different steps. The two stage (two-step) process is commonly known as B-staging.

Also, in the curing oven 60, the insulation pack 40 may be compressed by upper and lower foraminous oven conveyors 65, 70 to form a fibrous insulation blanket 10. It is to be appreciated that the insulation blanket 10 has an upper surface and a lower surface. In particular, the insulation blanket 10 has two major surfaces, typically a top and bottom surface, and two minor or side surfaces with fiber blanket 10 oriented so that the major surfaces have a substantially horizontal orientation. The upper and lower oven conveyors 65, 70 may be used to compress the insulation pack 40 to give the insulation blanket 10 a predetermined thickness. It is to be appreciated that although FIG. 1 depicts the conveyors 65, 70 as being in a substantially parallel orientation, they may alternatively be positioned at an angle relative to each other (not illustrated).

The curing oven 60 may be operated at a temperature from about 100° C. to about 325° C., or from about 250° C. to about 300° C. The insulation pack 40 may remain within the oven for a period of time sufficient to crosslink (cure) the binder and form the insulation blanket 10. A facing material 93 is then placed on the insulation blanket 10 to form a facing layer 95. Non-limiting examples of suitable facing materials 93 include Kraft paper, a foil-scrim-Kraft paper laminate, recycled paper, and calendared paper. The facing material 93 may be adhered to the surface of the insulation blanket 10 by a bonding agent (not shown) to form a faced insulation product 97. Suitable bonding agents include adhesives, polymeric resins, asphalt, and bituminous materials that can be coated or otherwise applied to the facing material 93. The faced fibrous insulation 97 may subsequently be rolled for storage and/or shipment or cut into predetermined lengths by a cutting device (not illustrated). Such faced insulation products may be used, for example, as panels in basement finishing systems, as ductwrap, ductboard, as faced residential insulation, and as pipe insulation. It is to be appreciated that, in some exemplary embodiments, the insulation blanket 10 that emerges from the oven 60 is rolled onto a take-up roll or cut into sections having a desired length and is not faced with a facing material 94.

A significant portion of the insulation placed in the insulation cavities of buildings is in the form of insulation blankets rolled from insulation products such as is described above. Faced insulation products are installed with the facing placed flat on the edge of the insulation cavity, typically on the interior side of the insulation cavity. Insulation products where the facing is a vapor retarder are commonly used to insulate wall, floor, or ceiling cavities that separate a warm interior space from a cold exterior space. The vapor retarder is placed on one side of the insulation product to retard or prohibit the movement of water vapor through the insulation product.

The presence of water, dust, and/or other microbial nutrients in the insulation product 10 may support the growth and proliferation of microbial organisms. Bacterial and/or mold growth in the insulation product may cause odor, discoloration, and deterioration of the insulation product 10, such as, for example, deterioration of the vapor barrier properties of the Kraft paper facing. To inhibit the growth of unwanted microorganisms such as bacteria, fungi, and/or mold in the insulation product 10, the insulation pack 40 may be treated with one or more anti-microbial agents, fungicides, and/or biocides. The anti-microbial agents, fungicides, and/or biocides may be added during manufacture or in a post manufacture process of the insulation product 10. It is to be appreciated that the insulation product using the inventive binder composition can be a fiberglass batt as depicted, or as loosefill insulation, ductboard, ductliner, or pipe wrap (not depicted in the Figures).

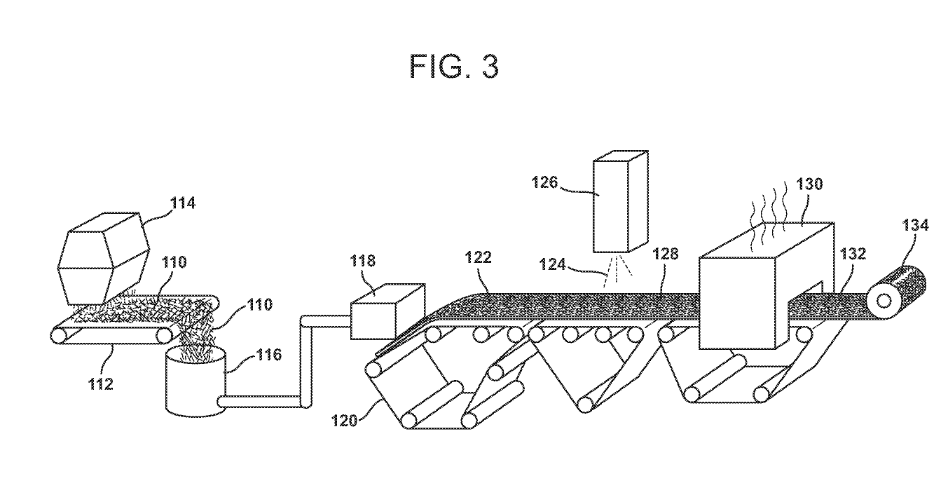

In a second embodiment of the present invention, the binder composition may be used to form a non-woven chopped strand mat. In particular, binder is added during the formation of the chopped strand mat in a wet-laid mat processing line. One exemplary process of separately adding the coupling agent to the chopped strand mat is depicted in FIG. 3. It is to be appreciated that reference is made herein to glass fibers, although the chopped strand mat could be formed of, or include, non-glass fibers. Chopped glass fibers 100 may be provided to a conveying apparatus such as a conveyor 112 by a storage container 114 for conveyance to a mixing tank 116 that contains various surfactants, viscosity modifiers, defoaming agents, and/or other chemical agents with agitation to disperse the fibers and form a chopped glass fiber slurry (not shown). The glass fiber slurry may be transferred to a head box 118 where the slurry is deposited onto a conveying apparatus such as a moving screen or foraminous conveyor 120 and a substantial portion of the water from the slurry is removed to form a web (mat) 122 of enmeshed fibers. The water may be removed from the web 122 by a conventional vacuum or air suction system (not shown).

The inventive binder 124 is applied to the web 122 by a suitable binder applicator, such as the spray applicator 126 or a curtain coater (not illustrated). Once the binder 124 has been applied to the mat 122, the binder coated mat 128 is passed through at least one drying oven 130 to remove any remaining water and cure the binder composition 124. The formed non-woven chopped strand mat 132 that emerges from the oven 130 is an assembly of randomly oriented, dispersed, individual glass fibers. The chopped strand mat 132 may be rolled onto a take-up roll 134 for storage for later use as illustrated. The non-woven mat can be use in roofing, flooring, ceiling, wall applications, as filters, in ground based vehicles, and in aircraft.

There are numerous advantages provided by the inventive binder formulation. For example, unlike conventional urea-formaldehyde binders, the binder formulation has a light (e.g., tan) color after curing. In addition, the modified starch is natural in origin and derived from renewable resources. Also, the binder composition can be cured at temperatures lower than conventional formaldehyde-based binders, thereby reducing manufacturing costs and gaseous emissions. By lowering or eliminating formaldehyde emission, the overall volatile organic compounds (VOCs) emitted in the workplace are reduced. Additionally, because modified starch compounds are relatively inexpensive, the insulation product or chopped fiber mat can be manufactured at a lower cost. The binder has low to no odor, making it more desirable to work with. Further, the binder permits the formed foamed product to be easily pigmented.

Having generally described this invention, a further understanding can be obtained by reference to certain specific examples illustrated below which are provided for purposes of illustration only and are not intended to be all inclusive or limiting unless otherwise specified.

### EXAMPLES

### Example 1

Samples of binder formulations according to Table 4 were prepared according to the following procedure. First, the crosslinker (i.e., citric acid, Kymene®, or QXRP 1734) was added to water in a first container. In a separate, second vessel, the starch was modified by the addition of suitable quantities of sulfuric acid. The modified starch dispersion was added to the crosslinker/water solution to form a stock mixture. The cure accelerator (i.e., sodium hypophosphite) and the silane (i.e., γ-aminopropyltriethoxysilane) were added to the stock solution to form the binder compositions.

TABLE 4

| | Total | Solids (%) | | | | | | |
|---|---|---|---|---|---|---|---|---|
| | (as | | | 40% | 20% | 100% | 38% | 52% |
| | received | | 2% | Sodium | Modified | Citric | Kymene ® | QRXP |
| Sample | (g)) | Water | Silane | Hypophosphite | Starch⁽¹⁾ | Acid | 736⁽²⁾ | 1734⁽³⁾ |
| 1 | 800 | 599 | 6.00 | 2.00 | 193 | | | |
| 2 | 800 | 622 | 6.00 | 2.00 | 164 | 5.69 | | |
| 3 | 800 | 613 | 6.00 | 2.00 | 164 | | 14.97 | |
| 4 | 800 | 617 | 6.00 | 2.00 | 164 | | | 10.88 |

⁽¹⁾a modified starch with a viscosity from 2-45 cps at 9% solids (from 200-625 cps at 22% solids)
⁽²⁾an aqueous solution of a cationic amine polymer-epichlorohydrin adduct (commercially available from Hercules Inc.)
⁽³⁾a polyacrylic acid resin (commercially available from The Dow Chemical Company)

The binder formulations set forth in Table 4 were then utilized to form handsheets in a manner known by those of skill in the art. The handsheets were dried and cured for three minutes at 450° F. The tensile strength, the LOI, and the tensile strength/LOI for each sample were determined under ambient and steam conditions. The results are set forth in Table 5.

TABLE 5

| | Hydration | | | | | |
|---|---|---|---|---|---|---|
| | | | Ambient | | | Steam |
| | | Ambient | Tensile | | Steam | Tensile |
| | | Tensile | Strength | | Tensile | Strength |
| | Ambient LOI | Strength | (lb/1 in)/ | Steam LOI | Strength | (lb/1 in)/ |
| Sample | (%) | (lb/1 in) | LOI (%) | (%) | (lb/1 in) | LOI (%) |
| 1 | 9 | 14.8 | 1.6 | 9 | 19.7 | 2.2 |
| 2 | 7 | 16.4 | 2.3 | 7.2 | 19.6 | 2.7 |
| 3 | 6.8 | 17.5 | 2.6 | 7.2 | 17.2 | 2.4 |
| 4 | 5.9 | 14.2 | 2.4 | 5.6 | 17.3 | 3.1 |

From Table 5, it was concluded that the addition of a crosslinker improved the tensile strength/LOI for the samples in both ambient and steam conditions.

### Example 2

Samples of binder formulations according to Table 6 were prepared according to the following procedure. First, the crosslinker (i.e., citric acid) was added to water in a first container. In a separate, second vessel, the starch was modified by the addition of suitable quantities of sulfuric acid. The modified starch dispersion was added to the crosslinker/water solution to form a stock mixture. The cure accelerator (i.e., sodium hypophosphite) and the silane (i.e., γ-aminopropyltriethoxysilane) were added to the stock solution to form the binder compositions.

TABLE 6

| | Total | | Solids (%) | | | |
|---|---|---|---|---|---|---|
| | (as | | | 40% | 100% | 20% |
| | received | | 2% | Sodium | Citric | Modified |
| Sample | (g)) | Water | Silane | Hypophosphite | Acid | Starch⁽¹⁾ |
| 1 | 800 | 601 | 6.04 | 1.91 | | 191 |
| 2 | 800 | 624 | 6.04 | 1.91 | 5.74 | 163 |
| 3 | 800 | 603 | 5.74 | | | 191 |
| 4 | 800 | 626 | 5.74 | | 5.72 | 162 |

⁽¹⁾a modified starch with a viscosity from 2-45 cps at 9% solids (from 200-625 cps at 22% solids)

The binder formulations set forth in Table 6 were then utilized to form handsheets in a manner known by those of skill in the art. The handsheets were dried and cured for three minutes at 450° F. The tensile strength, the LOI, and the tensile strength/LOI for each sample were determined under ambient and steam conditions. The results are set forth in Table 7.

TABLE 7

| | Hydration | | | | | |
|---|---|---|---|---|---|---|
| | | | Ambient | | | |
| | | Ambient | Tensile | | Steam | Steam |
| | | Tensile | Strength | Steam | Tensile | Tensile |
| | Ambient LOI | Strength | (lb/1 in)/ | LOI | Strength | Strength (lb/1 in)/ |
| Sample | (%) | (lb/1 in) | LOI (%) | (%) | (lb/1 in) | LOI (%) |
| 1 | 8.5 | 16.9 | 2.0 | 8 | 18.4 | 2.3 |
| 2 | 8.4 | 21.9 | 2.6 | 8 | 14.9 | 1.9 |
| 3 | 7.5 | 17 | 2.3 | 7.7 | 15.5 | 2 |
| 4 | 8 | 17.3 | 2.2 | 8.3 | 16 | 1.9 |

From Table 7, it was concluded that the addition of a crosslinker and a cure accelerator improved the tensile strength/LOI for the samples under ambient conditions. Additionally, it was concluded that the addition of the cure accelerator improved the tensile strength/LOI under steam conditions.

### Example 3

Samples of binder formulations according to Table 8 were prepared according to the following procedure. First, the crosslinker (i.e., triethanol amine, glycerol, citric acid, or QXRP 1734) was added to water in a first container. The modified starch dispersion (i.e., Super Film® 270W) was added to the crosslinker/water solution to form a stock mixture. The cure accelerator (i.e., sodium hypophosphite) and the silane (i.e., γ-aminopropyltriethoxysilane) were added to the stock solution to form the binder compositions.

TABLE 8

| | | | Solids (%) | | | | |
|---|---|---|---|---|---|---|---|
| | Total | | | | 18% | | |
| | (as | | | 40% | Super | | 50% |
| | received | | 2% | Sodium | Film ® | 100% | QRXP |
| Sample | (g)) | Water | Silane | Hypophosphite | 270W⁽¹⁾ | Crosslinker | 1734⁽²⁾ |
| 1 | 800 | 435 | 9.60 | | 356 | | |
| 2 | 800 | 435 | 9.60 | | 356 | | |
| 3 | 800 | 479 | 9.60 | | 302 | Triethanol | |
| | | | | | | Amine | |
| | | | | | | 9.6 | |
| 4 | 800 | 479 | 9.60 | | 302 | Glycerol | |
| | | | | | | 9.6 | |
| 5 | 800 | 479 | 9.60 | | 302 | Citric Acid | |
| | | | | | | 9.6 | |
| 6 | 800 | 469 | 9.60 | | 302 | | 19.2 |
| 7 | 800 | 518 | 7.80 | | 144 | | |
| 8 | 800 | 432 | 9.60 | 3.20 | 356 | | |

⁽¹⁾a modified starch with a viscosity from 2-45 cps at 9% solids (commercially available from Cargill)
⁽²⁾a polyacrylic acid resin (commercially available from The Dow Chemical Company)

The binder formulations set forth in Table 8 were then utilized to form handsheets in a manner known by those of skill in the art. The handsheets were dried and cured for three minutes at 400° F. The tensile strength, the LOI, and the tensile strength/LOI for each sample were determined under ambient and steam conditions. The results are set forth in Table 9.

TABLE 9

| | Hydration | | | | | |
|---|---|---|---|---|---|---|
| | | | Ambient | | | Steam |
| | | Ambient | Strength | | Steam | Tensile |
| | | Tensile | (lb/1 in)/ | Steam | Tensile | Strength |
| | Ambient LOI | Strength | LOI | Tensile LOI | Strength | (lb/1 in)/ |
| Sample | (%) | (lb/1 in) | (%) | (%) | (lb/1 in) | LOI (%) |
| 1 | 8.6 | 17.5 | 2 | 8.3 | 14.7 | 1.8 |
| 2 | 7.5 | 13.5 | 1.8 | 8.2 | 14.8 | 1.8 |
| 3 | 7.5 | 11 | 1.5 | 7.3 | 10.8 | 1.5 |
| 4 | 7.1 | 12.3 | 1.7 | 7.3 | 10.7 | 1.5 |
| 5 | 6.3 | 13.6 | 2.1 | 7.3 | 13.5 | 1.9 |
| 6 | 7.9 | 21.7 | 2.7 | 7.8 | 16.6 | 2.1 |
| 7 | 9.2 | 13.4 | 1.5 | 8.2 | 13.4 | 1.6 |
| 8 | 9.6 | 20.7 | 2.2 | 8.9 | 21.1 | 2.4 |

From Table 9, it was concluded that the addition of QXRP 1734 (i.e., a polyacrylic acid resin cure accelerator) provided a large increase in the tensile strength/LOI for the samples in both ambient and steam conditions.

### Example 4

Samples of binder formulations according to Table 10 were prepared according to the following procedure. First, the crosslinker was added to water in a first container. In Samples 1, 2, 3, 4, 6, 7, and 11, dilute sulfuric acid was added to lower the pH to 3 after the crosslinker was added. In Sample 5, the pH was adjusted to a pH of 5 after the addition of the crosslinker. The modified starch dispersion (i.e., Super Film® 270W) was added to the crosslinker/water solution to form a stock mixture. The cure accelerator and the silane (i.e., γ-aminopropyltriethoxysilane) were added to the stock solution to form the binder compositions.

For sample 12, the acetic anhydride and adipic acid were added directly to the starch dispersion. This mixture was permitted to stand for 30 minutes. The silane was then added.

TABLE 10

| | | | Solids (%) | | | | |
|---|---|---|---|---|---|---|---|
| | Total | | | | 18% | | |
| | (as | | | | Super | | |
| | received | | 2% | 40% | Film ® | 100% | 25% |
| Sample | (g)) | Water | Silane | Cure Accelerator | 270W⁽¹⁾ | Crosslinker | Crosslinker |
| 1 | 800 | 435 | 9.60 | | 356 | | |
| 2 | 800 | 479 | 9.60 | | 302 | Triethanol | |
| | | | | | | Amine | |
| | | | | | | 9.6 | |
| 3 | 800 | 475 | 9.60 | Sodium | 302 | Triethanol | |
| | | | | Hypophosphite | | Amine | |
| | | | | 3.20 | | 9.6 | |
| 4 | 800 | 432 | 9.60 | Trisodium | 356 | | |
| | | | | Tetrametaphosphate | | | |
| | | | | 3.2 | | | |
| 5 | 800 | 432 | 9.60 | Trisodium | 356 | | |
| | | | | Tetrametaphosphate | | | |
| | | | | 3.2 | | | |
| 6 | 800 | 479 | 9.60 | | 302 | Glycerol | |
| | | | | | | 9.6 | |
| 7 | 800 | 475 | 9.60 | Sodium | 302 | Glycerol | |
| | | | | Hypophosphite | | 9.6 | |
| | | | | 3.20 | | | |
| 8 | 800 | 453 | 9.60 | Sodium | 302 | Acumer | |
| | | | | Hypophosphite | | 9932⁽²⁾ | |
| | | | | 3.20 | | 32 | |
| 9 | 800 | 450 | 9.60 | | 302 | | Ammonium |
| | | | | | | | Citrate |
| | | | | | | | 38.4 |
| 10 | 800 | 475 | 9.60 | Sodium | 302 | Citric Acid | |
| | | | | Hypophosphite | | 9.6 | |
| | | | | 3.20 | | (neutralized | |
| | | | | | | with | |
| | | | | | | ammonium | |
| | | | | | | hydroxide) | |
| 11 | 800 | 432 | 9.60 | Sodium | 356 | | |
| | | | | Hypophosphite | | | |
| | | | | 3.20 | | | |
| 12 | 800 | 425 | 9.60 | | 356 | Adipic | |
| | | | | | | Acid | |
| | | | | | | 0.31 + | |
| | | | | | | Acetic | |
| | | | | | | Anhydride | |
| | | | | | | 9.3 | |

⁽¹⁾a modified starch with a viscosity from 2-45 cps at 9% solids (commercially available from Cargill)
⁽²⁾a 46% solids polyacrylic acid (commercially available from The Dow Chemical Company)

The binder formulations set forth in Table 10 were then utilized to form handsheets in a manner known by those of skill in the art. The handsheets were dried and cured for three minutes at 400° F. The tensile strength, the LOI, and the tensile strength/LOI for each sample were determined under ambient and steam conditions. The results are set forth in Table 11.

TABLE 11

| | Hydration | | | | | |
|---|---|---|---|---|---|---|
| | | | Ambient | | | |
| | | | Tensile | | | Steam |
| | | Ambient | Strength | | Steam | Tensile |
| | | Tensile | (lb/1 in)/ | | Tensile | Strength |
| | Ambient LOI | Strength | LOI | Steam LOI | Strength | (lb/1 in)/ |
| Sample | (%) | (lb/1 in) | (%) | (%) | (lb/1 in) | LOI (%) |
| 1 | 9.1 | 16.1 | 1.8 | 8.6 | 14.3 | 1.7 |
| 2 | 8.2 | 11.2 | 1.4 | 8.1 | 10.4 | 1.3 |
| 3 | 8.3 | 13.5 | 1.6 | 7.9 | 12.4 | 1.6 |
| 4 | 8.9 | 15.8 | 1.8 | 7.1 | 11.7 | 1.6 |
| 5 | 9.4 | 19.8 | 2.1 | 9.4 | 16.9 | 1.8 |
| 6 | 6.5 | 13.1 | 2.0 | 7.1 | 12 | 1.7 |
| 7 | 7.4 | 13.6 | 1.8 | 7.3 | 16.9 | 2.3 |
| 8 | 7.7 | 25 | 3.2 | 7.7 | 20.7 | 2.7 |
| 9 | 8.8 | 19.6 | 2.2 | 9.1 | 18.1 | 2 |
| 10 | 6.9 | 18.4 | 2.7 | 7.9 | 17.4 | 2.2 |
| 11 | 8.7 | 17.7 | 2.0 | 8.5 | 16.1 | 1.9 |
| 12 | 7.9 | 14.8 | 1.9 | 8.1 | 15.7 | 1.9 |

The following conclusions can be derived from Table 11:
The addition of trisodium trimetaphosphate increased the tensile strength/LOI when sulfuric acid was not added to reduce the pH
The addition of glycerol increased the ambient tensile strength/LOI of the samples
The addition of glycerol and sodium hypophosphite increased the tensile strength/LOI of the samples under steam conditions
The addition of Acumer 9932 and sodium hyphophosphite increased the tensile strength/LOI for the samples under both ambient and steam conditions
The addition of citric acid that had been neutralized with ammonium hydroxide increased the tensile strength/LOI of the samples
The addition of citric acid and sodium hypophosphite increased the tensile strength/LOI for the samples in ambient and steam conditions
The addition of sodium hypophosphite increased the tensile strength/LOI of the samples
The addition of adipic acid and acetic anhydride increased the tensile strength/LOI for the samples

### Example 5

Samples of binder formulations according to Table 12 were prepared according to the following procedure. First, the crosslinker was added to water in a first container. The modified starch dispersion (i.e., Super Film® 270W) was added to the crosslinker/water solution to form a stock mixture. The cure accelerator (i.e., sodium hypophosphite) and the silane (i.e., γ-aminopropyltriethoxysilane) were added to the stock solution to form the binder compositions.

TABLE 12

| | | | Solids (%) | | | |
|---|---|---|---|---|---|---|
| | | | | | 18% | |
| | Total | | | | Super | |
| | (as received | | 2% | 40% | Film ® | |
| Sample | (g)) | Water | Silane | Cure Accelerator | 270W⁽¹⁾ | Crosslinker |
| 1 | 800 | 435 | 9.60 | | 356 | |
| 2 | 800 | 479 | 9.60 | | 302 | Triethanol |
| | | | | | | Amine |
| | | | | | | 9.6 |
| | | | | | | (100%) |
| 3 | 800 | 471 | 9.60 | Sodium | 302 | Triethanol |
| | | | | Hypophosphite | | Amine |
| | | | | 8.0 | | 9.6 |
| | | | | | | (100%) |
| 4 | 800 | 456 | 9.60 | | 302 | Acumer 9932⁽²⁾ |
| | | | | | | 32 |
| | | | | | | (30%) |
| 5 | 800 | 471 | 9.60 | Sodium | 302 | Citric Acid |
| | | | | Hypophosphite | | 9.6 |
| | | | | 8.0 | | (100%) |
| 6 | 800 | 449 | 9.60 | | 320 | Acumer 9932⁽²⁾ |
| | | | | | | 21.33 |
| | | | | | | (30%) |
| 7 | 800 | 456 | 9.60 | Sodium | 320 | Citric Acid |
| | | | | Hypophosphite | | 6.4 |
| | | | | 8.0 | | (100%) |
| 8 | 800 | 441 | 9.60 | Sodium | 320 | Acumer 9932⁽²⁾ |
| | | | | Hypophosphite | | 21.33 |
| | | | | 8.0 | | (30%) |
| 9 | 800 | 448 | 9.60 | Sodium | 320 | Citric Acid |
| | | | | Hypophosphite | | 6.4 |
| | | | | 16.0 | | (100%) |
| 10 | 800 | 442 | 9.60 | | 338 | Acumer 9932⁽²⁾ |
| | | | | | | 10.67 |
| | | | | | | (30%) |
| 11 | 800 | 441 | 9.60 | Sodium | 338 | Citric Acid |
| | | | | Hypophosphite | | 3.2 |
| | | | | 8.0 | | (100%) |
| 12 | 800 | 442 | 9.60 | Sodium | 302 | Ammonium |
| | | | | Hypophosphite | | Citrate |
| | | | | 8.0 | | 38.4 |
| | | | | | | (25%) |
| 13 | 800 | 442 | | Sodium | 302 | Sodium Citrate |
| | | | | Hypophosphite | | 38.4 |
| | | | | 8.0 | | (25%) |
| 14 | 800 | 449 | 9.6 | | 302 | QXRP 1734⁽³⁾ |
| | | | | | | 19.2 |
| | | | | | | (50%) |

⁽¹⁾a modified starch with a viscosity from 2-45 cps at 9% solids (commercially available from Cargill)
⁽²⁾a 46% solids polyacrylic acid (commercially available from The Dow Chemical Company)
⁽³⁾a polyacrylic acid resin (commercially available from The Dow Chemical Company)

The binder formulations set forth in Table 12 were then utilized to form handsheets in a manner known by those of skill in the art. The handsheets were dried and cured for three minutes at 400° F. The tensile strength, the LOI, and the tensile strength/LOI for each sample were determined under both ambient and steam conditions. The results are set forth in Table 13.

TABLE 13

| | | Hydration | | | | | |
|---|---|---|---|---|---|---|---|
| | | | | Ambient | | | |
| | | | | Tensile | | | Steam |
| | | | Ambient | Strength | | Steam | Tensile |
| | | | Tensile | (lb/1 in)/ | Steam | Tensile | Strength |
| | | Ambient | Strength | LOI | LOI | Strength | (lb/1 in)/ |
| Sample | Final pH | LOI (%) | (lb/1 in) | (%) | (%) | (lb/1 in) | LOI (%) |
| 1 | 3 | 10.5 | 16.9 | 1.6 | 10.5 | 19.1 | 1.8 |
| 2 | 3 | 9.3 | 8.7 | 0.9 | 8.1 | 6.6 | 0.8 |
| 3 | 3 | 9.4 | 17.1 | 1.8 | 9.2 | 18.6 | 2.0 |
| 4 | 3.2 | 7.8 | 21.8 | 2.8 | 9.0 | 19.6 | 2.2 |
| 5 | 2.6 | 7.3 | 19.7 | 2.7 | 8.3 | 14.8 | 1.8 |
| 6 | 3.4 | 9.3 | 21.8 | 2.3 | 9.8 | 24.3 | 2.5 |
| 7 | 2.7 | 8.0 | 18.1 | 2.3 | 8.3 | 20.3 | 2.4 |
| 8 | 3.4 | 9.1 | 29.3 | 3.2 | 9.4 | 25.6 | 2.7 |
| 9 | 2.8 | 8.2 | 20.3 | 2.5 | 8.7 | 18.5 | 2.1 |
| 10 | 3.7 | 10.3 | 22.5 | 2.2 | 10 | 22.8 | 2.3 |
| 11 | 3 | 8.5 | 20 | 2.4 | 8.7 | 16.6 | 1.9 |
| 12 | 7 | 9.9 | 25.7 | 2.6 | 10 | 25.1 | 2.5 |
| 13 | 6.9 | 9.9 | 14 | 1.4 | 10 | 12.1 | 1.2 |
| 14 | 3.2 | 8.7 | 21.8 | 2.5 | 8.9 | 19.6 | 2.2 |

The following conclusions can be derived from Table 13:
The addition of Acumer 9932 increased the tensile strength/LOI of the samples under both ambient and steam conditions
The addition of citric acid and sodium hypophosphite to the samples increased both the ambient and steam tensile strength/LOI
The addition of Acumer 9932 and sodium hypophosphite to the samples increased the tensile strength/LOI under both ambient and steam conditions
The addition of citric acid and twice the amount of sodium hypophosphite (i.e., 16.0 vs. 8.0 at 40%) increased the ambient tensile strength/LOI of the sample
The addition of 5% Acumer 9932 increased ambient and steam tensile strength/LOI of the sample
The addition of sodium hypophosphite and 5% Acumer 9932 to the sample increased the tensile strength/LOI under both ambient and steam conditions
The addition of ammonium citrate and sodium hypophosphite increased the tensile strength/LOI of the samples under both ambient and steam conditions
The addition of QXRP 1734 to the sample increased both the ambient and steam tensile strength/LOI

### Example 6

Samples of binder formulations according to Table 14 were prepared according to the following procedure. First, the crosslinker was added to water in a first container. The modified starch dispersion (i.e., Super Film® 270W) was added to the crosslinker/water solution to form a stock mixture. The cure accelerator (i.e., sodium hypophosphite) and the silane (i.e., γ-aminopropyltriethoxysilane) were added to the stock solution to form the binder compositions.

TABLE 14

| | | | Solids (%) | | | | | | |
|---|---|---|---|---|---|---|---|---|---|
| | Total | | | | 18% | | | | |
| | (as | | | 40% | Super | 30% | 25% | 100% | 30% |
| | received | | 2% | Sodium | Film ® | Acumer | Ammonium | Malto- | Acumer |
| | (g)) | Water | Silane | Hypophosphite | 270W⁽²⁾ | 9932⁽³⁾ | Citrate | dextrin | 9932⁽³⁾ |
| 1 | 800 | 467 | 9.00 | 3.75 | 300 | 20 | | | |
| 2 | 800 | 464 | 9.00 | 7.50 | 300 | 20 | | | |
| 3 | 1400 | 824 | 15.75 | | 525 | 35 | | | |
| 4 | 800 | 464 | 9.00 | | 317 | 10 | | | |
| 5 | 800 | 457 | 9.00 | 7.50 | 317 | 10 | | | |
| 6 | 800 | 464 | 9.00 | 7.50 | 300 | | | | 20 |
| 7 | 800 | 470 | 9.00 | 7.50 | 283 | | | | 30 |
| 8 | 800 | 460 | 9.00 | 7.50 | 300 | | 24 | | |
| 9 | 800 | 467 | 9.00 | | 300 | | 24 | | |
| 10 | 800 | 710 | 9.00 | 7.50 | | 20 | | 54 | |
| 11 | 920 | 795 | 12.42 | 10.35 | | 27.6 | | 75 | |

⁽¹⁾neutralized with NH₄O₄
⁽²⁾a modified starch with a viscosity from 2-45 cps at 9% solids (commercially available from Cargill)
⁽³⁾a 46% solids polyacrylic acid (commercially available from The Dow Chemical Company)

The binder formulations set forth in Table 14 were then utilized to form handsheets in a manner known by those of skill in the art. The handsheets were dried and cured for three minutes at 400° F. The tensile strength, the LOI, and the tensile strength/LOI for each sample were determined under both ambient and steam conditions. The results are set forth in Table 15

TABLE 15

| | | Hydration | | | | | |
|---|---|---|---|---|---|---|---|
| | | | | Ambient | | | |
| | | | | Tensile | | | Steam |
| | | | Ambient | Strength | | Steam | Tensile |
| | | | Tensile | (lb/1 in)/ | Steam | Tensile | Strength |
| | | Ambient | Strength | LOI | LOI | Strength | (lb/1 in)/ |
| Sample | Final pH | LOI (%) | (lb/1 in) | (%) | (%) | (lb/1 in) | LOI (%) |
| 1 | 3.5 | 7.9 | 19.7 | 2.5 | 7.8 | 18.6 | 2.4 |
| 2 | 3.6 | 7.7 | 19.6 | 2.6 | 8 | 20.8 | 2.6 |
| 3 | 3.5 | 7.7 | 18.9 | 2.5 | 7.9 | 15.1 | 1.9 |
| 4 | 3.8 | 7.3 | 15.2 | 2.1 | 7.5 | 16.5 | 2.2 |
| 5 | 3.9 | 7.2 | 16.8 | 2.3 | 7.4 | 17.4 | 2.3 |
| 6 | 6.8 | 8.3 | 20.4 | 2.5 | 8.4 | 16.6 | 2 |
| 7 | 6.9 | 7.7 | 23.3 | 3.0 | 8.2 | 19.5 | 2.4 |
| 8 | 6.7 | 8 | 20.3 | 2.5 | 7.8 | 18.2 | 2.3 |
| 9 | 6.6 | 7.1 | 17.2 | 2.4 | 8.6 | 16.6 | 1.9 |
| 10 | 2.8 | 5.3 | 10 | 1.9 | 5.6 | 11.2 | 2 |
| 11 | 2.9 | 5.3 | 10.5 | 2.0 | 5.1 | 6.6 | 1.3 |

Although a standard starch at pH 3 (control) was not included in this example, by comparing the results of this example to Examples 1-5 above, it was concluded that Samples 1-9 demonstrated an ambient and steam tensile strength/LOI that was better than the control systems.

### Example 7

Binder formulations formed of starch/polyacrylic acid triethanolamine (PAT) and starch/citric acid were prepared in the ratios and components set forth in Table 16. The binder formulations set forth in Table 16 were then utilized to form R-19 fiberglass insulation batts in a conventional manner known by those of skill in the art. These inventive binders were compared to a conventional phenolic binder with respect to dead ambient thickness, recovered thickness at ambient conditions, and ambient stiffness. The results are set forth in Table 17.

TABLE 16

| | | Oil | Starch | QRXP | Soy | Citric | |
|---|---|---|---|---|---|---|---|
| | | Emulsion | Premix | 1734 | Premix | Acid | Y15612 |
| | | (% solids) | (% solids) | (% solids) | (% solids) | (% solids) | (% solids) |
| | | 50% | 18% | 50% | 20% | 100% | 40% |
| | | Oil | Starch | QRXP | Soy | Citric | |
| | | Emulsion | Premix | 1734 | Premix | Acid | Y15612 |
| | | (wt as | (wt as | (wt as | (wt as | (wt as | (wt as |
| | Description | received) | received) | received) | received) | received) | received) |
| 1 | 85:15 Starch- | 7.4% | 87.0% | 5.5% | 0.0% | 0.0% | 0.1% |
| | PAT | | | | | | |
| 2 | 70:30 Starch- | 8.2% | 79.4% | 12.3% | 0.0% | 0.0% | 0.2% |
| | PAT | | | | | | |
| 3 | 50:50 Starch- | 5.4% | 49.7% | 0.0% | 44.8% | 0.0% | 0.1% |
| | Soy | | | | | | |
| 4 | 90:10 Soy- | 6.0% | 0.0% | 4.0% | 89.9% | 0.0% | 0.1% |
| | PAT | | | | | | |
| 5 | 85:15 Starch- | 7.6% | 89.4% | 0.0% | 0.0% | 2.8% | 0.1% |
| | Citric Acid | | | | | | |
| 6 | 85:15 Starch- | 4.4% | 92.5% | 0.0% | 0.0% | 2.9% | 0.2% |
| | Citric Acid | | | | | | |
| | (TRS) | | | | | | |

TABLE 17

| | | Recovered | |
|---|---|---|---|
| Property | Dead Thickness | Thickness | Stiffness |
| Tested | (Ambient) | (Ambient) | (Ambient) |
| Test Method | ASTM C167 | ASTM C167 | S-05Ec |
| Unit | Inches | Inches | degrees |
| 1 | 5.52 | 6.27 | 33.15 |
| 2 | 5.54 | 6.26 | 31.44 |
| 3 | 5.48 | 6.52 | 43.99 |
| 4 | 5.34 | 6.68 | 48.93 |
| 5 | 5.50 | 6.31 | 38.96 |
| 6 | 5.65 | 6.25 | 30.13 |
| Conventional | 5.67 | 6.32 | 30.29 |
| Phenolic | | | |
| Binder | | | |

As shown in Tables 16 and 17, the starch based binders demonstrated improved recovered thickness and stiffness at ambient conditions compared to conventional phenolic binders.

The invention of this application has been described above both generically and with regard to specific embodiments. Although the invention has been set forth in what is believed to be the preferred embodiments, a wide variety of alternatives known to those of skill in the art can be selected within the generic disclosure. The invention is not otherwise limited, except for the recitation of the claims set forth below.

Having thus described the invention, what is claimed is:

1. An aqueous binder composition for use in the formation of fiberglass insulation and non-woven mats comprising:
at least one modified starch natural in origin and having a degree of polymerization from about 20 to about 4000; and
at least one silane coupling agent.

2. The aqueous binder composition of claim 1, wherein:
said modified starch is derived from a plant source selected from corn, potatoes, soybeans, rice, beets, sugar cane, cassava and mixtures thereof; and
said silane coupling agent is selected from aminosilanes, epoxy trialkoxysilanes, methyacryl trialkoxysilanes, hydrocarbon trialkoxysilanes, amino trihydroxysilanes, epoxy trihydroxysilanes, methacryl trihydroxy silanes, hydrocarbon trihydroxysilanes and combinations thereof.

3. The aqueous binder composition of claim 1, further comprising at least one member selected from a crosslinking agent, a dust suppression agent, a cure accelerator, a pH adjusting agent and a moisture resistant agent.

4. The aqueous binder composition of claim 3, wherein said binder includes one or more members selected from a crosslinking agent and a cure accelerator.

5. The aqueous binder of claim 4, wherein said crosslinking agent is selected from citric acid and salts thereof, polyacrylic acid and salts thereof, polyacrylic acid resin, triethanolamine, sodium metaborate, polyoxyalkyleneamines, polyamines, glycerol, triethanolamine, polyols, polyacrylic acid, polycarboxylic acid, polycarboxylic acid with anhydride, organic acids, inorganic acids, organic bases, inorganic bases, proteins and combinations thereof.

6. The aqueous binder composition of claim 4, wherein said cure accelerator is selected from sodium hypophosphite, sodium phosphate, potassium phosphate, disodium pyrophosphate, tetrasodium pyrophosphate, sodium tripolyphosphate, sodium hexamethaphosphate, potassium phosphate, potassium tripolyphosphate, sodium trimetaphosphate, sodium tetramethaphosphate and mixtures thereof.

7. The aqueous binder composition of claim 4, wherein said binder further includes a pH adjusting agent, said pH adjusting the pH of said binder composition to a pH from about 1 to about 6.

8. The aqueous binder composition of claim 1, wherein said modified starch has a viscosity less than about 205 cps at 9% solids.

9. A fibrous insulation product comprising:
a plurality of randomly oriented fibers; and
a binder composition applied to at least a portion of said fibers and interconnecting said fibers, said binder composition including:
at least one modified starch derived from natural sources and having a degree of polymerization from about 20 to about 4000; and
at least one silane coupling agent.

10. The fibrous insulation product of claim 9, wherein said modified starch has a viscosity less than about 205 cps at 9% solids.

11. The fibrous insulation product of claim 9, wherein:
said modified starch is derived from a plant source selected from corn, potatoes, soybeans, rice, beets, sugar cane, cassava and mixtures thereof; and
said silane coupling agent is selected from aminosilanes, epoxy trialkoxysilanes, methyacryl trialkoxysilanes, hydrocarbon trialkoxysilanes, amino trihydroxysilanes, epoxy trihydroxysilanes, methacryl trihydroxy silanes, hydrocarbon trihydroxysilanes and combinations thereof.

12. The fibrous insulation product of claim 9, further comprising at least one member selected from a crosslinking agent, a dust suppression agent, a cure accelerator, a pH adjusting agent and a moisture resistant agent.

13. The fibrous insulation product of claim 9, wherein said binder includes one or more members selected from a crosslinking agent and a cure accelerator.

14. The fibrous insulation product of claim 13, wherein said binder further includes a pH adjusting agent, said pH adjusting the pH of said binder composition to a pH from about 1 to about 6.

15. A non-woven chopped strand mat comprising:
a plurality of randomly oriented glass fibers having a discrete length enmeshed in the form of a mat having a first major surface and a second major surface; and
a binder composition at least partially coating said first major surface of said mat, said binder composition including:
at least one modified starch derived from natural sources and having a degree of polymerization from about 20 to about 4000; and
at least one silane coupling agent.

16. The non-woven chopped strand mat of claim 15, further comprising at least one member selected from a crosslinking agent, a dust suppression agent, a cure accelerator, a pH adjusting agent and a moisture resistant agent.

17. The non-woven chopped strand mat of claim 16, wherein said binder includes one or more members selected from a crosslinking agent, a cure accelerator and a pH adjusting agent.

18. The non-woven chopped strand mat of claim 17, wherein said crosslinking agent is selected from citric acid and salts thereof, polyacrylic acid and salts thereof, polyacrylic acid resin, triethanolamine, sodium metaborate, polyoxyalkyleneamines, polyamines, glycerol, triethanolamine, polyols, polyacrylic acid, polycarboxylic acid, polycarboxylic acid with anhydride, organic acids, inorganic acids, organic bases, inorganic bases, proteins and combinations thereof,
wherein said cure accelerator is selected from sodium hypophosphite, sodium phosphate, potassium phosphate, disodium pyrophosphate, tetrasodium pyrophosphate, sodium tripolyphosphate, sodium hexamethaphosphate, potassium phosphate, potassium tripolyphosphate, sodium trimetaphosphate, sodium tetramethaphosphate and mixtures thereof, and
wherein said pH adjusting the pH of said binder composition to a pH from about 1 to about 6.

19. The non-woven chopped strand mat of claim 16, wherein said binder composition has a light color after curing.

20. The non-woven chopped strand mat of claim 16, wherein:
said modified starch is derived from a plant source selected from corn, potatoes, soybeans, rice, beets, sugar cane, cassava and mixtures thereof; and
said silane coupling agent is selected from aminosilanes, epoxy trialkoxysilanes, methyacryl trialkoxysilanes, hydrocarbon trialkoxysilanes, amino trihydroxysilanes, epoxy trihydroxysilanes, methacryl trihydroxy silanes, hydrocarbon trihydroxysilanes and combinations thereof.
